(19) United States
(12) Patent Application Publication
(10) Pub. No.: US 2023/0046186 A1
(43) Pub. Date: Feb. 16, 2023

(54) Process for the preparation of angiotensin receptor blockers or pharmaceutically acceptable salts thereof

(71) Applicant: Jubilant Pharma Holdings Inc., Yardley, PA (US)

(72) Inventors: Saji Thomas, Noida (IN); Rajendra Shekhawat, Noida (IN); M. Umamaheshwar Prasad, Noida (IN); Bidyut Biswas, Noida (IN); Rohit Chakravarthy, Noida (IN); Chetan Balubhai Patel, Nanjangud (IN); Anilkumar Haribhat Lingabhat, Nanjangud (IN); Mohan Chikmagalur Sadashivappa, Nanjangud (IN); Indranil Nandi, Yardley, PA (US)

(73) Assignee: Jubilant Pharma Holdings Inc., Yardley, PA (US)

(21) Appl. No.: 17/739,862

(22) Filed: May 9, 2022

Related U.S. Application Data
(63) Continuation-in-part of application No. 17/459,374, filed on Aug. 27, 2021, now Pat. No. 11,327,065.
Application No. 17/459,374 is a continuation-in-part of application No. 17/409,003, filed on Aug. 23, 2021.
(60) Provisional application No. 63/226,991, filed on Jul. 29, 2021.

(30) Foreign Application Priority Data
Jul. 29, 2021 (IN) 202111034277

(51) Int. Cl. C07D 403/10; A61K 31/4178

(52) U.S. Cl. CPC C07D 403/10; A61K 31/4178

(57) ABSTRACT

The present invention relates to a process for the preparation of angiotensin receptor blockers or its pharmaceutically acceptable salts thereof containing less than 10 ppm of the azido impurities. More particularly, the present invention relates to process for the preparation of Losartan, Losartan potassium of Formula I or its other pharmaceutically acceptable salts thereof containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole. More particularly, the present invention relates to a simple, economical and industrially efficient process for the preparation of Losartan potassium of Formula I. The present invention also relates to solid oral pharmaceutical compositions and process for preparing solid oral pharmaceutical compositions comprising losartan or its pharmaceutically acceptable salts thereof and method of detecting azido impurities in these solid oral pharmaceutical compositions.

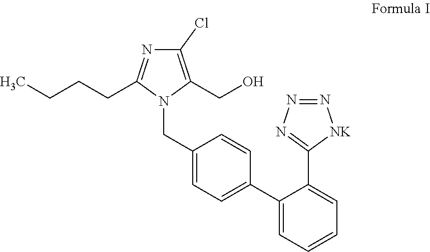

### FIELD OF THE INVENTION

The present invention relates to a process for the preparation of angiotensin receptor blockers or its pharmaceutically acceptable salts thereof. More particularly, the present invention relates to a simple, economical and industrially efficient process for the preparation of Losartan potassium of Formula I.

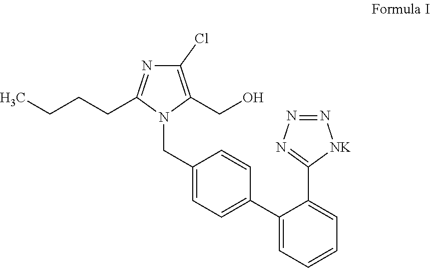

### BACKGROUND OF THE INVENTION

The chemical name for Losartan is (2-butyl-4-chloro-1-{[2′-(2H-tetrazol-5-yl)biphenyl-4-yl]methyl}-1H-imidazol-5-yl)methanol. The CAS Registry Number of Losartan is [114798-26-4], which has the following structure.

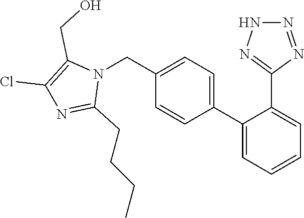 [SMILES: CCCCc1nc(Cl)c(CO)n1Cc1ccc(-c2ccccc2-c2nn[nH]n2)cc1]

Losartan and its potassium salt are angiotensin-II receptor (Type AT1) antagonists. In adults, Losartan is currently indicated for the treatment of hypertension. The incidence of hypertension is very high with every third person in the world is suffering from this condition.

This provides a huge market potential for antihypertensive drugs. Due to its high market value over the world, cost effective synthesis of Losartan is highly desirable.

U.S. Pat. No. 5,138,069 discloses and claims Losartan, its derivatives and pharmaceutically acceptable salts, including the potassium salt, as well as a method of treatment using pharmaceutically acceptable salts of Losartan. This patent also discloses a process for the preparation of Losartan and its derivatives, which comprises de-protecting trityl-Losartan with hydrochloric acid to form free base of Losartan, and then adding aqueous potassium hydroxide-isopropanol solution to convert the free base to its potassium salt.

The known processes suffer from problems of purity of Losartan or its pharmaceutically acceptable salts thereof regarding presence of undesirable carcinogenic azido impurities. The processes disclosed in the prior art fail to provide the control of undesirable carcinogenic/genotoxic azido impurities.

Azido impurities such as 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole, 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole, which are highly mutagenic/genotoxic/carcinogenic and are point of concern.

Consequently, there is a need for an improved process for the preparation of Losartan or its pharmaceutically acceptable salts thereof, which not only overcomes the problems in the prior art processes as mentioned above, but also is simple, economically viable, industrially feasible and environment friendly for the preparation of Losartan or its pharmaceutically acceptable salts thereof having a good control over carcinogenic/genotoxic azido impurities and its carcinogenic precursor impurities.

In view of the same, there is a need for simple, industrially feasible, cost effective and environmentally-friendly process for the preparation of Losartan or its pharmaceutically acceptable salts thereof free from carcinogenic/genotoxic azido impurities and its carcinogenic precursor impurities.

The problem has been solved by providing an improved process, wherein Losartan or its pharmaceutically acceptable salts thereof is heated in the presence of solvent with aqueous alkali to isolate pure Losartan or its pharmaceutically acceptable salts thereof, which is free from carcinogenic/genotoxic azido impurities and its carcinogenic precursor impurities.

### OBJECT OF THE INVENTION

It is a principal object of the present invention to improve upon limitations of the prior arts by providing an efficient process for the preparation of Losartan and/or pharmaceutically acceptable salts thereof.

It is another object of the present invention to provide a simple, commercially viable, economical and environment friendly process for preparing Losartan and/or pharmaceutically acceptable salts thereof.

The present invention relates to a process for the preparation of angiotensin receptor blockers namely Losartan, Valsartan, Candesartan, Olmesartan or Irbesartan or its pharmaceutically acceptable salts thereof containing less than 10 ppm of the azido impurities.

It is still another object of the present invention to provide an improved process for the preparation of Losartan or its pharmaceutically acceptable salts thereof containing less than 10 ppm of each of the carcinogenic azido impurity, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole.

It is still another object of the present invention to provide Losartan or its pharmaceutically acceptable salts thereof containing less than 10 ppm of each of the undesirable carcinogenic azido impurity, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole.

It is still another object of the present invention to provide an improved process for the preparation of Losartan or its pharmaceutically acceptable salts thereof containing less than 10 ppm of each of the carcinogenic azido impurity by heating Losartan or its pharmaceutically acceptable salts thereof in solvent with aqueous alkali to isolate Losartan or its pharmaceutically acceptable salts thereof free from carcinogenic azido impurities.

It is still another object of the present invention to provide an improved process for the preparation of Losartan or Losartan potassium containing less than 10 ppm of each of the carcinogenic azido impurity by heating Losartan or Losartan potassium in solvent with aqueous alkali to isolate Losartan or Losartan potassium free from carcinogenic azido impurities.

It is still another object of the present invention to provide an improved process for the preparation of Losartan or Losartan potassium containing less than 10 ppm of each of the carcinogenic azido impurity by treating Losartan or Losartan potassium with an alkali in presence of solvent and reagent and/or gas or mixture thereof to isolate Losartan or Losartan potassium free from carcinogenic azido impurities.

It is still another object of the present invention to provide an improved process for the preparation of Losartan or Losartan potassium containing less than 10 ppm of each of the carcinogenic azido impurity by heating Losartan or Losartan potassium in solvent with aqueous alkali in presence of reagent and/or gas or mixture thereof to isolate Losartan or Losartan potassium free from carcinogenic azido impurities.

### SUMMARY OF THE INVENTION

The present invention relates to a process for the preparation of angiotensin receptor blockers namely Losartan, Valsartan, Candesartan, Olmesartan or Irbesartan or its pharmaceutically acceptable salts thereof containing less than 10 ppm of the azido impurities.

According to one aspect of the present invention there is provided an efficient and cost effective process for the preparation of Losartan or its pharmaceutically acceptable salts thereof containing less than 10 ppm of each of the carcinogenic azido impurity, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole.

It has been found that Losartan or its pharmaceutically acceptable salts thereof containing less than 10 ppm of each of the carcinogenic azido impurity is efficiently prepared by heating Losartan or its pharmaceutically acceptable salts thereof in solvent with aqueous alkali.

It is still another object of the present invention to provide an improved process for the preparation of Losartan or Losartan potassium containing less than 10 ppm of each of the carcinogenic azido impurity by treating Losartan or Losartan potassium with an alkali in presence of solvent and reagent and/or gas or mixture thereof to isolate Losartan or Losartan potassium free from carcinogenic azido impurities.

It has been found that Losartan or its pharmaceutically acceptable salts thereof containing less than 10 ppm of each of the carcinogenic azido impurity is efficiently prepared by heating Losartan or its pharmaceutically acceptable salts thereof in solvent with aqueous alkali in presence of reagent and/or gas or mixture thereof.

Heating Losartan or its pharmaceutically acceptable salts thereof, preferably potassium salt, in solvent with aqueous alkali helps in achieving Losartan or its pharmaceutically acceptable salts thereof containing less than 10 ppm of each of the carcinogenic azido impurity.

### DESCRIPTION OF THE INVENTION

The present invention relates to a process for the preparation of angiotensin receptor blockers namely Losartan, Valsartan, Candesartan, Olmesartan or Irbesartan or its pharmaceutically acceptable salts thereof containing less than 10 ppm of the each of the azido impurities.

Azido impurities of Losartan or Losartan potassium are highly mutagenic/genotoxic/carcinogenic and are point of concern during the synthesis of angiotensin receptor blockers.

These azido impurities are:
5-(4′-(azidomethyl)-[1,1′-biphenyl]-2-yl)-1H-tetrazole of Formula II
4′-(azidomethyl)-[1,1′-biphenyl]-2-carbonitrile of Formula III,
4′-((5-(azidomethyl)-2-butyl-4-chloro-1H-imidazol-1-yl)methyl)-[1,1′-biphenyl]-2-carbonitrile of Formula IV,
5-(4′-((5-(azidomethyl)-2-butyl-4-chloro-1H-imidazol-1-yl)methyl)-[1,1′-biphenyl]-2-yl)-1H-tetrazole of Formula V,
5-(azidomethyl)-2-butyl-4-chloro-1H-imidazole of Formula VI,
4′-((4-(azidomethyl)-2-butyl-5-chloro-1H-imidazol-1-yl)methyl)-[1,1′-biphenyl]-2-carbonitrile of Formula VII,
5-(4′-((4-(azidomethyl)-2-butyl-5-chloro-1H-imidazol-1-yl)methyl)-[1,1′-biphenyl]-2-yl)-1H-tetrazole of Formula VIII and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole of Formula IX.

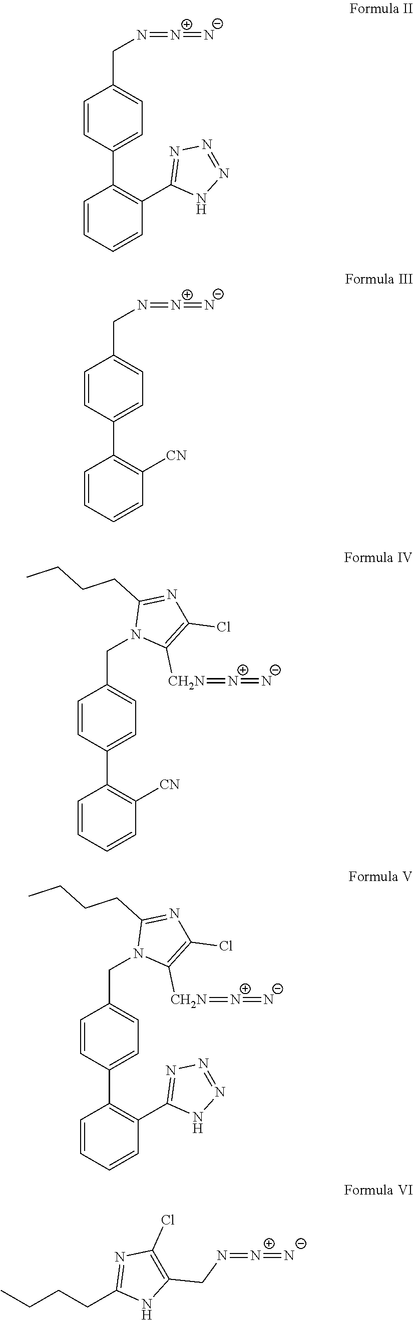

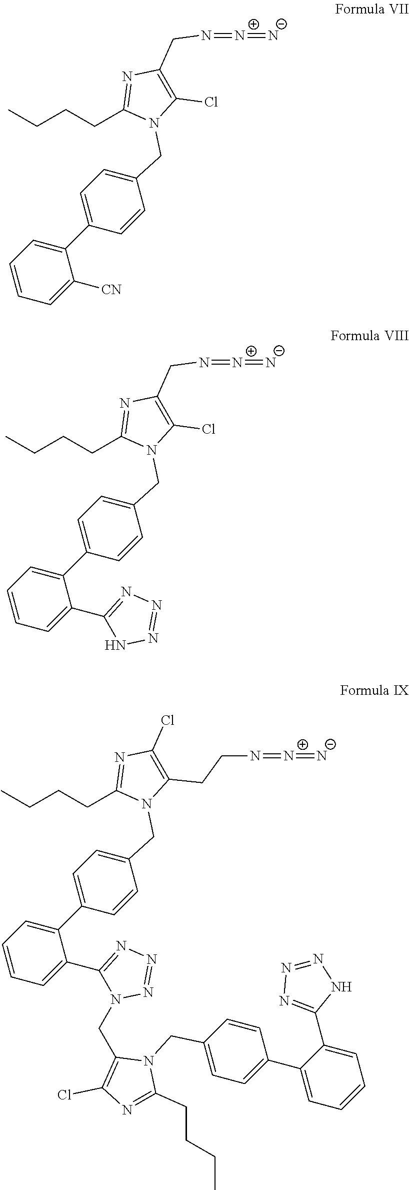 [SMILES: CCCCc1nc(CN=[N+]=[N-])c(Cl)n1Cc1ccc(-c2ccccc2C#N)cc1 ; CCCCc1nc(CN=[N+]=[N-])c(Cl)n1Cc1ccc(-c2ccccc2-c2nnn[nH]2)cc1 ; CCCCc1nc(Cl)c(CCN=[N+]=[N-])n1Cc1ccc(-c2ccccc2-c2nnnn2Cc2c(Cl)nc(CCCC)n2Cc2ccc(-c3ccccc3-c3nnn[nH]3)cc2)cc1]

In an embodiment, the present invention relates to a process for the preparation of Losartan or its pharmaceutically acceptable salts thereof containing less than 10 ppm of each of the azido impurity, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
(i) providing a solution of Losartan or its pharmaceutically acceptable salts thereof in solvent with aqueous alkali;
(ii) heating the reaction mixture; and
(iii) isolating Losartan or its pharmaceutically acceptable salts thereof.

In step (i), solvent is selected from the group comprising of water, organic solvent and mixtures thereof. The organic solvent is selected from the group comprising of hydrocarbon solvent, polar aprotic solvent, polar protic solvent, non-polar solvent and the like. The organic solvent is further selected from the group comprising of pentane, hexane, 1,4-dioxane, diethyl ether, tetrahydrofuran (THF), ethyl acetate, toluene, xylene, acetone, dimethylformamide (DMF), acetonitrile (ACN), methanol, ethanol, propanol, n-butanol and the like.

In step (i), the alkali is selected from the group comprising of sodium hydroxide, potassium hydroxide, lithium hydroxide, calcium hydroxide and magnesium hydroxide. Alkali in step (i) is preferably sodium hydroxide or potassium hydroxide.

In step (i), the concentration of aqueous sodium or potassium hydroxide solution is selected from 0.5% to 35%. Concentration of aqueous sodium or potassium hydroxide solution is preferably 2.5% to 5.0%.

In step (i), mole equivalents of alkali with respect to Losartan is from 0.1 to 5 mole equivalents.

Alternatively, potassium iodide, sodium iodide and the like can also be used along with aqueous sodium hydroxide solution for depletion of azido impurities.

Alternatively reducing agents such as sodium borohydride, lithium borohydride and the like can also be used along with an aqueous base solution for depletion of the azido impurities.

In step (ii), heating is carried out at temperature from 40° C. to 110° C. Heating in step (ii) is carried out preferably at temperature from 90° C. to 100° C.

In step (ii), heating is carried out for 8 hours to 24 hours, preferably 10 hours to 15 hours.

In step (iii), isolation is performed by conventional means already known in the prior art which includes is but not limited to filtration, distillation, crystallization, acid-base treatment and the like, preferably filtration.

As per instant invention, the Limit of Detection (LOD) for these azido impurities is 1 ppm.

In an embodiment, the present invention relates to a process for the preparation of Losartan or its pharmaceutically acceptable salts thereof containing less than 10 ppm of each of the azido impurity, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl) methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl) methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azido methyl)-2-butyl-4-chloro-1H-imidazol-1-yl)methyl)-[1,1′-biphenyl]-2-yl)-1H-tetrazole comprising the steps of:
(i) providing a solution of Losartan or its pharmaceutically acceptable salts thereof in solvent with aqueous alkali;
(ii) heating the reaction mixture;
(iii) isolating Losartan or its pharmaceutically acceptable salts thereof; and
(iv) optionally repetition of steps (i) to (iii), to obtain Losartan or its pharmaceutically acceptable salts thereof having azido impurity less than 10 ppm.

In an embodiment, the present invention relates to a process for the preparation of Losartan or its pharmaceutically acceptable salts thereof containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl) methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azido methyl)-2-butyl-4-chloro-1H-imidazol-1-yl)methyl)-[1,1′-biphenyl]-2-yl)-1H-tetrazole comprising the steps of:
(i) providing a solution of Losartan or its pharmaceutically acceptable salts thereof in solvent with aqueous alkali;
(ii) heating the reaction mixture;
(iii) adding organic solvent and adjusting pH 3 to 5 by means of adding acid to the reaction mixture;
(iv) filtering the reaction mixture; and
(v) isolating Losartan or its pharmaceutically acceptable salts thereof.

In step (ii), heating is carried out at temperature from 40° C. to 110° C., preferably at temperature from 90° C. to 100° C.

In step (ii), heating is carried out for 8 hours to 24 hours, preferably 10 hours to 15 hours.

In an another embodiment, the present invention relates to a process for the preparation of Losartan potassium containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
(i) providing a solution of Losartan potassium in water with potassium hydroxide;
(ii) heating the reaction mixture to 90° C. to 100° C.; and
(iii) isolating Losartan potassium containing less than 10 ppm of each of the azido impurities.

In an another embodiment, the present invention relates to a process for the preparation of Losartan or Losartan potassium containing less than 10 ppm of 5-(4′-((5-(azidomethyl)-2-butyl-4-chloro-1H-imidazol-1-yl)methyl)-[1,1′-biphenyl]-2-yl)-1H-tetrazole of Formula V, comprising the steps of:
(i) providing a solution of Losartan or Losartan potassium in solvent with aqueous alkali;
(ii) heating the solution at a temperature from 40° C. to 110° C. for 8 hrs to 24 hrs; and
(iii) isolating Losartan or Losartan potassium.

In an embodiment, the present invention relates to a process for the preparation of Losartan or its pharmaceutically acceptable salts thereof containing less than 10 ppm of each of the azido impurity, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
(i) providing a solution of Losartan or its pharmaceutically acceptable salts thereof in solvent with alkali;
(ii) heating the reaction mixture in presence of reagent and/or gas or mixture thereof; and
(iii) isolating Losartan or its pharmaceutically acceptable salts thereof.

In step (i), solvent is selected from the group comprising of water, organic solvent and mixtures thereof. The organic solvent is selected from the group comprising of hydrocarbon solvent, polar aprotic solvent, polar protic solvent, non-polar solvent and the like. The organic solvent is further selected from the group comprising of pentane, hexane, 1,4-dioxane, diethyl ether, tetrahydrofuran (THF), ethyl acetate, toluene, xylene, acetone, dimethylformamide (DMF), acetonitrile (ACN), methanol, ethanol, propanol, n-butanol and the like.

In step (i), the alkali is selected from the group comprising of sodium hydroxide, potassium hydroxide, lithium hydroxide, calcium hydroxide and magnesium hydroxide. Alkali in step (i) is preferably sodium hydroxide or potassium hydroxide.

The alkali in step (i) is preferably aqueous solution of alkali.

In step (i), the concentration of aqueous sodium or potassium hydroxide solution is selected from 0.5% to 35%. Concentration of aqueous sodium or potassium hydroxide solution is preferably 2.5% to 20%.

In step (i), mole equivalents of alkali with respect to Losartan is from 0.1 to 5 mole equivalents.

In step (ii), reagent is any hydrogen source which facilitates in reducing azido impurity and preferably less than 10 ppm.

In step (ii), reagent is selected from the group comprising of sodium borohydride, sodium borohydride with phase transfer catalyst, lithium borohydride, zinc borohydride, lithium aluminium hydride, glucose, ammonium formate with zinc, ammonium formate with copper, alkyldichloro borane, aryldichloro borane, triethyl borane, borontrifluoride diethyl etherate with sodium iodide or potassium iodide and the like.

In step (ii), gas is selected from the group comprising of hydrogen gas, nitrogen gas, argon gas, neon gas and the like. Preferably, said reaction in presence of gas is performed in autoclave/hydrogenator under pressure.

An Autoclave is a machine used to carry out industrial and scientific processes requiring elevated temperature and pressure in relation to ambient pressure and/or temperature.

In step (ii), said reaction in presence of gas is performed without metal catalyst in autoclave/hydrogenator under pressure.

In step (ii), when gas is used, pressure of gas is at least 2 Kg/cm².

In step (ii), heating is carried out at temperature from 70° C. to 110° C. Heating in step (ii) is carried out preferably at temperature from 80° C. to 100° C.

In step (ii), heating is carried out for 10 min to 10 hrs, preferably 2 hrs to 8 hrs and more preferably 1 hr to 4 hrs.

In step (iii), isolation is performed by conventional means already known in the prior art which includes is but not limited to filtration, distillation, crystallization, acid-base treatment and the like, preferably filtration.

In an embodiment, the present invention relates to a process for the preparation of Losartan or its pharmaceutically acceptable salts thereof containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl) methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azido methyl)-2-butyl-4-chloro-1H-imidazol-1-yl)methyl)-[1,1′-biphenyl]-2-yl)-1H-tetrazole comprising the steps of:
(i) providing a solution of Losartan or its pharmaceutically acceptable salts thereof in solvent with alkali;
(ii) heating the reaction mixture in presence of reagent and/or gas or mixture thereof;
(iii) adding organic solvent and adjusting pH 3 to 5 by means of adding acid to the reaction mixture;
(iv) filtering the reaction mixture; and
(v) isolating Losartan or its pharmaceutically acceptable salts thereof.

In step (ii), reagent is selected from the group comprising of sodium borohydride, sodium borohydride with phase transfer catalyst, lithium borohydride, zinc borohydride, lithium aluminium hydride, glucose, ammonium formate with zinc, ammonium formate with copper, alkyldichloro borane, aryldichloro borane, triethyl borane, borontrifluoride diethyl etherate with sodium iodide or potassium iodide.

In step (ii), gas is selected from the group comprising of hydrogen gas, nitrogen gas and the like.

In step (ii), heating is carried out at temperature from 70° C. to 110° C. Heating in step (ii) is carried out preferably at temperature from 80° C. to 100° C.

In step (ii), heating is carried out 10 min to 10 hrs, preferably 2 hrs to 8 hrs and more preferably 1 hr to 4 hrs.

In an embodiment, the present invention relates to a process for the preparation of Losartan or its pharmaceutically acceptable salts thereof containing less than 10 ppm of any individual azido impurity, wherein the azido impurity is selected from the group consisting of:
5-(4′-(azidomethyl)-[1,1′-biphenyl]-2-yl)-1H-tetrazole,
4′-(azidomethyl)-[1,1′-biphenyl]-2-carbonitrile,
4′-((5-(azidomethyl)-2-butyl-4-chloro-1H-imidazol-1-yl)methyl)-[1,1′-biphenyl]-2-carbonitrile,
5-(4′-((5-(azido methyl)-2-butyl-4-chloro-1H-imidazol-1-yl)methyl)-[1,1′-biphenyl]-2-yl)-1H-tetrazole,
5-(azidomethyl)-2-butyl-4-chloro-1H-imidazole,
4′-((4-(azidomethyl)-2-butyl-5-chloro-1H-imidazol-1-yl)methyl)-[1,1′-biphenyl]-2-carbonitrile,
5-(4′-((4-(azidomethyl)-2-butyl-5-chloro-1H-imidazol-1-yl)methyl)-[1,1′-biphenyl]-2-yl)-1H-tetrazole, and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
   (i) providing a solution of Losartan or its pharmaceutically acceptable salts thereof in a solvent with an alkali;
   (ii) heating the reaction mixture in an autoclave; and
   (iii) isolating Losartan or its pharmaceutically acceptable salts thereof.

When hydrogen gas is used in an autoclave, then said heating reaction is carried out for 10 min to 10 hrs, preferably 2 hrs to 8 hrs and more preferably 1 hr to 4 hrs.

When nitrogen gas is used in an autoclave, then said heating reaction is carried out for 10 min to 10 hrs, preferably 4 hrs to 8 hrs and more preferably 6 hrs to 8 hrs.

In step (ii), heating is carried out at temperature from 70° C. to 110° C. Heating in step (ii) is carried out preferably at temperature from 80° C. to 100° C.

In an another embodiment, the present invention relates to a process for the preparation of Losartan potassium containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
(i) providing a solution of Losartan or Losartan potassium in water with potassium hydroxide;
(ii) heating the reaction mixture with glucose; and
(iii) isolating Losartan potassium containing less than 10 ppm of each of the azido impurities.

In step (ii), heating is carried out at temperature from 70° C. to 110° C. Heating in step (ii) is carried out preferably at temperature from 80° C. to 100° C.

In an another embodiment, the present invention relates to a process for the preparation of Losartan potassium containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
(i) providing a solution of Losartan or Losartan potassium in water with potassium hydroxide;
(ii) heating the reaction mixture with sodium borohydride and tetra butyl ammonium bromide; and
(iii) isolating Losartan potassium containing less than 10 ppm of each of the azido impurities. Wherein tetra butyl ammonium bromide is used in catalytic amount.

In step (ii), heating is carried out at temperature from 70° C. to 110° C. Heating in step (ii) is carried out preferably at temperature from 80° C. to 100° C.

In an another embodiment, the present invention relates to a process for the preparation of Losartan potassium containing less than 10 ppm of each of the azido impurities, wherein the azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
(i) providing a solution of Losartan or Losartan potassium in water with alkali;
(ii) heating the reaction mixture in presence of reagent and/or gas or mixture thereof;
(iii) adding organic solvent and adjusting the pH to a pH of 3 to 5 by means of adding acid to the reaction mixture;
(iv) filtering the reaction mixture of step (iii); and
(v) isolating Losartan potassium containing less than 10 ppm of each of the azido impurities.

In an another embodiment, the present invention relates to a process for the preparation of Losartan or its pharmaceutically acceptable salts thereof containing less than 10 ppm of any individual azido impurity, wherein the azido impurity is selected from the group consisting of:
5-(4′-(azidomethyl)-[1,1′-biphenyl]-2-yl)-1H-tetrazole,
4′-(azidomethyl)-[1,1′-biphenyl]-2-carbonitrile,
4′-((5-(azidomethyl)-2-butyl-4-chloro-1H-imidazol-1-yl)methyl)-[1,1′-biphenyl]-2-carbonitrile,
5-(4′-((5-(azidomethyl)-2-butyl-4-chloro-1H-imidazol-1-yl)methyl)-[1,1′-biphenyl]-2-yl)-1H-tetrazole,
5-(azidomethyl)-2-butyl-4-chloro-1H-imidazole,
4′-((4-(azidomethyl)-2-butyl-5-chloro-1H-imidazol-1-yl)methyl)-[1,1′-biphenyl]-2-carbonitrile,
5-(4′-((4-(azidomethyl)-2-butyl-5-chloro-1H-imidazol-1-yl)methyl)-[1,1′-biphenyl]-2-yl)-1H-tetrazole, and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole comprising the steps of:
   (i) providing a solution of Losartan or its pharmaceutically acceptable salts thereof in a solvent with an alkali;
   (ii) heating the reaction mixture in presence of a reagent, a gas or a mixture thereof; and
   (iii) isolating Losartan or its pharmaceutically acceptable salts thereof, wherein the reagent is selected from the group comprising of glucose or sodium borohydride with tetra butyl ammonium bromide; and the gas is selected from the group comprising of hydrogen gas, nitrogen gas, argon and neon gas.

ICH M7 recommends that these mutagenic carcinogens be controlled at or below the acceptable cancer risk level. Due to their known potent carcinogenic effects, and because it is feasible to limit these impurities by taking reasonable steps to control or eliminate their presence, the goal is to have no quantifiable carcinogenic impurities or well within the declared limits which is safe for human consumption.

It is utmost important to control or deplete these undesirable carcinogenic azido impurities such as 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azido methyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole below 10 ppm in Losartan or its pharmaceutically acceptable salts thereof.

In an another embodiment, the present invention relates to a process for the preparation of Losartan potassium containing less than 10 ppm of each of the azido impurity.

It has been found that purification techniques like dissolving Losartan or Losartan potassium in water and washing with organic solvent such as methyl iso-butyl ketone, dichloromethane, heptane, toluene or ethyl acetate did not efficiently remove the carcinogenic azido impurities.

TABLE 1
Depletion of mutagenic azido impurity by dissolving Losartan
potassium in water and washing with organic solvent

| | Azido impurity of | |
|---|---|---|
| | Formula V | |
| | (ppm) | |
| | | After |
| Process | Initial | washing |
| Dissolving Losartan potassium in 3 V of purified | 315 | 190 |
| water and washing with methyl isobutyl ketone | | |
| Dissolving Losartan potassium in 3 V of purified | 315 | 169 |
| water and washing with dichloromethane | | |
| Dissolving Losartan potassium in 3 V of purified | 315 | 197 |
| water and washing with n-heptane | | |
| Dissolving Losartan potassium in 3 V of purified | 315 | 190 |
| water and washing toluene | | |
| Dissolving Losartan potassium in 3 V of purified | 315 | 188 |
| water and washing with ethyl acetate | | |

The present applicant has found that desired limit of azido impurities was not achieved when aqueous solution of Losartan potassium was extracted with organic solvents.

TABLE 2
Depletion of mutagenic azido impurity by
crystallization of Losartan potassium

| | Initial Azido impurity of | Final Azido impurity of |
|---|---|---|
| Solvent | Formula II content (ppm) | Formula II content (ppm) |
| IPA/n-Heptane | 428 | 157.5 |
| IPA | 204 | 137.25 |
| Ethyl acetate | 452.19 | 278.3 |

TABLE 3
Depletion of mutagenic azido impurity
by crystallization of Losartan

| | | | | Azido impurity |
|---|---|---|---|---|
| | | | Azido impurity | of Formula V |
| Batch | | | of Formula V | content after |
| Size | | | content in | crystallization |
| (g) | Solvent | Output(g) | input (ppm) | (ppm) |
| 4.0 | Ethyl acetate | 3.5 | 802.32 | 657 |
| 4.0 | Methyl isobutyl | 3.6 | 802.32 | 657 |
| | ketone | | | |
| 4.0 | Isopropyl alcohol | 2.0 | 802.32 | 657 |
| 30.0 | Ethyl acetate/water/ | 26 | 802.32 | 638 |
| | HCl | | | |
| 5.0 | Isopropyl alcohol/ | 3.0 | 802.32 | 638 |
| | water | | | |
| 5.0 | Ethyl acetate | 2.5 | 802.32 | 638 |

The present applicant has found that desired limit of azido impurities was not achieved by crystallization of Losartan or Losartan potassium using different solvents and/or mixture thereof.

TABLE 4
Content of various azido impurities in Losartan
while heating with Potassium carbonate
To Losartan, 5 volumes of 20% of aqueous Potassium
carbonate was added and it was heated at
95° C.-100° C. for 14 hr.

| Azido impurity - input (before treatment) | Azido impurity after treatment |
|---|---|
| 1845.8 ppm | 1838.5 ppm |

The present applicant has found that there was no depletion of azido impurity in Losartan when heated in aqueous potassium carbonate solution.

TABLE 5
Content of various azido impurities in Losartan
potassium while heating with Potassium carbonate
To Losartan potassium, 5 volumes of 20% of aqueous
Potassium carbonate was added and it was heated at
95° C.-100° C. for 14 hr.

| Azido impurity - input (before treatment) | Azido impurity after treatment |
|---|---|
| 601.7 ppm | 617.3 ppm |

The present applicant has found that there was no depletion of azido impurity in Losartan potassium when heated in aqueous potassium carbonate solution.

TABLE 6
Content of various azido impurities in Losartan and Losartan
potassium by employing the process of the instant invention
Azido impurity of Formula V content (in ppm)

| Input | Azido impurity | After heating in aqueous potassium |
|---|---|---|
| Batch | during reaction | hydroxide solution for 10-12 h at 95- |
| size | monitoring by HPLC | 100° C., followed by isolation of Losartan |
| 50 g | 3324 ppm | Less than 1 ppm |
| 50 g | 3019 ppm | Less than 1 ppm |

The problem has been solved by providing an improved process in which Losartan or its pharmaceutically acceptable salts thereof in solvent with aqueous alkali is heated and Losartan or its pharmaceutically acceptable salts thereof containing less than 10 ppm of each of the azido impurity is isolated.

The analytical method used for the determination of azido impurities in Losartan or pharmaceutically acceptable salts thereof is either LCMS/LCMS-MS method or HPLC method. The analytical method used for the determination of azido impurities during reaction monitoring is HPLC method. The analytical method used for the determination of azido impurities in isolated Losartan or its pharmaceutically acceptable salts thereof is LCMS or LCMS-MS method.

Specifically, analytical method for the determination of azido impurities in Losartan or its pharmaceutically acceptable salts thereof is LCMS or LCMS-MS method, wherein azido impurity is selected from the group comprising of 5-(4′-(azidomethyl)-[1,1′-biphenyl]-2-yl)-1H-tetrazole, 4′-(azidomethyl)-[1,1′-biphenyl]-2-carbonitrile, 4′-((5-(azidomethyl)-2-butyl-4-chloro-1H-imidazol-1-yl)methyl)-[1,1′-biphenyl]-2-carbonitrile, 5-(4′-((5-(azidomethyl)-2-butyl-4-chloro-1H-imidazol-1-yl)methyl)-[1,1′-biphenyl]-2-yl)-1H-tetrazole, 5-(azidomethyl)-2-butyl-4-chloro-1H-imidazole, 4′-((4-(azidomethyl)-2-butyl-5-chloro-1H-imidazol-1-yl)methyl)-[1,1′-biphenyl]-2-carbonitrile, 5-(4′-((4-(azidomethyl)-2-butyl-5-chloro-1H-imidazol-1-yl)methyl)-[1,1′-biphenyl]-2-yl)-1H-tetrazole and 1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole.

LCMS-MS method: Azido impurities in Losartan or Losartan potassium was determined by LCMS-MS method using ESI mode with mobile phase.

Mobile phase (water/methanol): 720 mL/1280 mL methanol and 0.1% formic acid Using Column: Xbridge C8 150×4.6, 5μ, Flow rate 0.8 mL/min

HPLC method: Azido impurities in Losartan or Losartan potassium was determined by HPLC method with mobile phase 10 mM phosphate buffer and 0.1% OPA, column Xterra RP18, 250×4.5, 5μ column temperature 40° C., isocratic method with buffer and methanol.

The process for the preparation of Losartan or its pharmaceutically acceptable salts thereof as described in the present invention is demonstrated in the examples illustrated below. Certain specific aspects and embodiments of the present application are explained in greater detail with reference to the following examples, which are provided only for purposes of illustration and should not be construed as limiting the scope of the disclosure in any manner. Reasonable variations of the described procedures are intended to be within the scope of the present application. While particular aspects of the present application have been illustrated and described, it would be apparent to those skilled in the art that various other changes and modifications can be made without departing from the spirit and scope of the disclosure. It is therefore intended to encompass all such changes and modifications that are within the scope of this disclosure.

### EXAMPLES

### Example-1: Preparation of (1-((2′-(1H-Tetrazol-5-yl)(1,1′-biphenyl)4-yl)methyl)-2-butyl-4-chloro-1H-imidazol-5-yl)methanol

Tetrazole ring formation of 4′-{[2-Butyl-4-chloro-5-(hydroxymethyl)-1H-imidazole-1-yl] methyl} biphenyl-2-carbonitrile (100 g) was performed using sodium azide (2.98 mole equivalent), triethylamine hydrochloride (3.30 mole equivalent), triethylamine (1.08 mole equivalent) and methyl isobutyl ketone (1.5V) as solvent. The reaction mass was heated at 95° C.-110° C. and maintained for 30 hr-35 hr. After reaction, the organic layer was extracted with aqueous Potassium hydroxide solution. To the aqueous layer containing Losartan, 20% of Potassium hydroxide was added and this solution was heated to 95° C.-100° C. and stirred it for 12 hr-14 hr. After charcoalization of the aqueous layer, pH was adjusted to 3.8-4.2 with addition of dilute HCl in presence of ethyl acetate and stirred for 6 hr-7 hr at 0° C.-5° C. Next, the product was filtered, isolated and dried. The Losartan was further purified by heating in 3.9% KOH solution (5 V) at 95° C.-100° C. for 12 hr-14 hr followed by isolation by adjusting the pH to 3.8-4.2 with the addition of dilute HCl in the presence of ethyl acetate and stirred for 6 hr-7 hr at 0° C.-5° C. The product then was filtered, isolated and dried. Azido impurity <1 ppm obtained from approximately 5,000-10,000 ppm formed during tetrazole reaction

### Example-2: Preparation of (1-((2′-(1H-Tetrazol-5-yl)(1,1′-biphenyl)4-yl)methyl)-2-butyl-4-chloro-1H-imidazol-5-yl)methanol

Tetrazole ring formation of 4′-{[2-Butyl-4-chloro-5-(hydroxymethyl)-1H-imidazole-1-yl] methyl} biphenyl-2-carbonitrile (100 g) was performed using sodium azide (2.98 mole equivalent), triethylamine hydrochloride (3.30 mole equivalent), tetrabutyl ammonium bromide and methyl isobutyl ketone (5V) as solvent and with the reaction mass heated at 95° C.-110° C. and maintained for 30 hr-35 hr. After completion of the reaction, the product was extracted into aqueous layer using 10% aqueous sodium hydroxide solution and this aqueous solution was heated to 12 hr-24 hr at 95° C.-100° C. till the azido impurities wear below 10 ppm. The Losartan is isolated after the pH is adjusted to 3.8-4.2 with addition of dilute HCl in the presence of ethyl acetate and stirred for 6 hr-7 hr at 0-5° C. The product then is filtered and isolated. The product is dried initially at 25° C.-30° C. for 2 hr under vacuum and then at 60° C.-65° C. for 8 hr under vacuum.

Molar Yield: 90%; HPLC Purity >99.0%

Azido impurity: 8 ppm obtained from 5,000-10,000 ppm formed during tetrazole reaction

### Example-3: Preparation of Potassium 5-[4′-[[2-butyl-4-chloro-5-(hydroxymethyl)-1H-imidazol-1-yl]methyl]biphenyl-2-yl]tetrazol-1-ide (Losartan Potassium Salt Formation)

Charged IPA (8V) and 50% solution of KOH at 25° C.-30° C. Stirred the resulting mixture for 30 min to get a clear solution at 25° C.-30° C. Charged Losartan (100 g) at 25° C.-30° C. Stirred the reaction mass for 1 hr to get clear solution at 25° C.-30° C. Check pH (pH 9.5-10.0). Charged activated carbon (2.7%) at 25° C.-30° C. Stirred the reaction mass for 30 min at 25° C.-30° C. Filtered the resulting mixture through hyflo bed washed with IPA (2V). Distilled out the solvent to approximately 70%, atmospherically below 85° C. Cooled the reaction mass to 60° C.-65° C. under nitrogen. Charged n-Heptane (1V) at 60° C.-65° C. under nitrogen. Gradually cooled the reaction mass to 25° C.-30° C. in 2 hr-3 hr under nitrogen. Stirred the reaction mass at 25° C.-30° C. for 6 hr-7 hr under nitrogen. Gradually cooled the reaction mass in 2 hr-3 hr to 0-5° C. under nitrogen. Stirred the reaction mass at 0° C.-5° C. for 2 hr under nitrogen. Then the reaction mass was filtered and washed with chilled IPA (1V) under nitrogen. Suck dried and unloaded the wet product under nitrogen. Dried the product initially at 25° C.-30° C. for 2 hr and then at 60° C.-65° C. for 8 hr under vacuum.

Molar Yield: 90%; HPLC Purity >99.5%

Azido impurity <1 ppm.

### Example-4: Purification of Losartan Potassium

Charged 3.9% KOH solution (19.5 g KOH dissolved in DM water (500 mL). Charged Losartan potassium (100 g). Heated to 95° C.-100° C. and stirred for 12 hr-20 hr. Cooled reaction mixture to 10° C.-15° C. Charged ethyl acetate (300 mL) at 10° C.-15° C. Slowly adjusted the pH to 3.8-4.2 with dilute HCl. Raised temperature of reaction mass to 25° C.-30° C. Resulting mixture was stirred for 6 hr-7 hr at 25° C.-30° C. Cooled the reaction mass to 0° C.-5° C. and stirred for 2 hr at 0-5° C. Filtered it and washed with DM water (3×120 mL) and then with ethyl acetate (120 mL) at 25° C.-30° C. Suck dried the filtered product and unloaded wet product. Dried the product initially at 25° C.-30° C. under vacuum for 2 hr and then at 60° C.-65° C. under vacuum for 8 hr.

Molar Yield: 90%; HPLC Purity >99.5%

Azido Impurity <1 ppm;

### Example-5: Purification of Losartan Potassium

Charged 3.9% KOH solution (19.5 g KOH dissolved in DM water (500 mL) and Losartan potassium (100 g). Heated to 95° C.-100° C. and stirred for 12 hr-20 hr. Cooled the reaction mixture to 10° C.-15° C. Charged ethyl acetate (300 mL) at 10° C.-15° C. Slowly adjusted the pH to 3.8-4.2 with dilute HCl. Raised temperature of reaction mass to 25° C.-30° C. Stirred resulting mixture for 6 hr-7 hr at 25° C.-30° C. Cooled reaction mass to 0° C.-5° C. and stirred for 2 hr at 0° C.-5° C. Filtered the material and washed with DM water (3×120 mL) and then ethyl acetate (120 mL) at 25° C.-30° C. Suck dried the filtered material and unloaded wet material. Dried the product initially at 25° C.-30° C. under vacuum for 2 hr and then at 60° C.-65° C. under vacuum for 8 hr till LOD is NMT 0.5%. Azido Impurity <1 ppm obtained from 200-500 ppm Losartan potassium.

### Example-6: Purification of Losartan Potassium

Charged IPA (8V) and 50% solution of KOH at 25° C.-30° C. Stirred the resulting mixture for 30 min to get clear solution at 25° C.-30° C. Charged Losartan potassium (100 g) at 25° C.-30° C. Stirred the reaction mass for 1 hr to get clear solution at 25° C.-30° C. Check pH (pH 9.5-10.0). Charged activated carbon (2.7%) at 25-30° C. Stirred the reaction mass for 30 min at 25-30° C. Filtered the resulting mixture through hyflo and hyflo bed washed with IPA (2V). The solvent was distilled out to approximately 70% atmospherically below 85° C. Cooled the reaction mass to 60° C.-65° C. under nitrogen. Charged n-Heptane (1V) at 60° C.-65° C. under nitrogen. Gradually cooled the reaction mass to 25° C.-30° C. in 2-3 hr under nitrogen. Stirred the reaction mass at 25° C.-30° C. for 6 hr-7 hr under nitrogen. Gradually cooled the reaction mass in 2 hr-3 hr to 0° C.-5° C. under nitrogen. Stirred the reaction mass at 0° C.-5° C. for 2 hr under nitrogen. Filtered the reaction mass and washed with IPA (1V) under nitrogen. Sucked dried and unloaded the wet material under nitrogen. Dried the product initially at 25° C.-30° C. for 2 hr and then at 60° C.-65° C. for 8 hr under vacuum.

Molar Yield: 85%; HPLC Purity >99.5%

Azido impurity <1 ppm

### Example-7: Purification of Losartan Potassium

Charged DM water (500 mL) and Losartan potassium (100 g) at 25° C.-30° C. A 20% aq. KOH solution (1.6 g KOH dissolved in DM water 8 mL) was added at 25° C.-30° C. Heated the reaction mixture to 95° C.-100° C. for 10 hr-12 hr. Distilled out DM water under vacuum below 80° C. till stir-able mass. Cooled the reaction mixture to 40° C. and added IPA (900 mL) and stirred for 15 min-30 min. Filtered the resulting mixture through hyflo bed, followed by filtration and washed with IPA (100 mL) at 40° C.-50° C. Charged clear filtrate into reactor at 25° C.-30° C. Distilled out IPA (˜700 mL) atmospherically below 85° C. (maintained the residual volume˜250 mL in the reactor). Cooled the reaction mass to 60° C.-65° C. under nitrogen. Charged n-heptane (50 mL) at 60° C.-65° C. under nitrogen. Gradually cooled the reaction mixture at 25° C.-30° C. in 2 hr-3 hr under nitrogen. Stirred the reaction mixture at 25° C.-30° C. for 6 hr-7 hr under nitrogen. Gradually cooled the reaction mixture in 2 hr-3 hr to 0° C.-5° C. under nitrogen. Stirred the reaction mixture at 0° C.-5° C. for 2 hr under nitrogen. Filtered the reaction mixture and washed with pre-filtered chilled (0° C.-5° C.) IPA (100 mL) under nitrogen. Sucked dried and unloaded the wet material under nitrogen. Dried the material initially at 25° C.-30° C. under vacuum for 2 hr and then at 60° C.-65° C. under vacuum for 8 hr.

Molar Yield: 90%; HPLC Purity >99.5%

Azido impurity <1 ppm obtained from 200-500 ppm Losartan Potassium.

### Example-8: Purification of Losartan Free Base

Charged Losartan free base (15.0 g) (having azido impurity at 2100-2500 ppm) in aqueous sodium hydroxide solution (2.5 g NaOH in 100 mL water) and heated to 1-2 hr at 75-80° C. under H₂ pressure in autoclave. Then pH was adjusted to 3.8-4.2 using aqueous HCl in presence of ethyl acetate (45 mL) and product was isolated. Dried the material initially at 25-30° C. for 2 h under vacuum and then at 60-65° C. for 8 h under vacuum.

Molar Yield: 90%; HPLC Purity >98.0%

Azido impurity: <1 ppm (BDL) obtained from 2100-2500 ppm azido impurity in Losartan Base.

### Example-9: Preparation of (1-((2′-(1H-Tetrazol-5-yl)(1,1′-biphenyl)4-yl)methyl)-2-butyl-4-chloro-1H-imidazol-5-yl)methanol

Tetrazole ring formation of 4′-{[2-Butyl-4-chloro-5-(hydroxymethyl)-1H-imidazole-1-yl] methyl} biphenyl-2-carbonitrile (100 g) is performed using sodium azide (51.0 g), triethylamine hydrochloride (145 g), Tetrabutyl ammonium bromide (7.0 g) in methyl isobutyl ketone (500 mL) and reaction mass heated at 95° C.-110° C. and maintained for 30 hr-35 hr. After completion of the reaction, the product was extracted into aqueous layer using 10% aqueous sodium hydroxide solution and this aqueous solution was heated to 1 hr-2 hr at 75° C.-85° C. under hydrogen pressure in an autoclave in absence of any metal catalyst. The Losartan is isolated after the pH is adjusted to 3.8-4.2 with addition of Aq. HCl in the presence of ethyl acetate. The product is dried initially at 25° C.-30° C. for 2 hr under vacuum and then at 60° C.-65° C. for 8 hr under vacuum.

Molar Yield: 90%; HPLC Purity >98.0%

Azido impurity: <1 ppm (BDL) obtained from 4000 to 5000 ppm formed during tetrazole reaction.

### Example-10: Purification of Losartan Free Base

Charged Losartan free base (5.0 g) (having azido impurity at 2100-2500 ppm) in aqueous Potassium hydroxide (KOH 0.80 g in 25 mL DM water) and glucose (1.06 g). Then the aqueous solution is heated to 2 hrs at 95° C.-100° C. in sealed tube. The product is isolated after the pH is adjusted to 3.8-4.2 with addition of Aq. HCl in the presence of ethyl acetate. The product is dried initially at 25° C.-30° C. for 2 hr under vacuum and then at 60° C.-65° C. for 8 hr under vacuum.

Molar Yield: 90%; HPLC Purity >98.0%

Azido impurity: <1 ppm (BDL) obtained from Losartan base having 2100-2500 ppm azido impurity.

### Example-11: Purification of Losartan Free Base

Charged Losartan free base (15.0 g) (having azido impurity at 2100-2500 ppm) in aqueous sodium hydroxide solution (2.5 g NaOH in 100 mL water) and the aqueous solution is heated to 10 hrs at 95° C.-100° C. under N₂ pressure in autoclave. The product is isolated after the pH is adjusted to 3.8-4.2 with addition of Aq. HCl in the presence of ethyl acetate. The product is dried initially at 25° C.-30° C. for 2 hr and then at 60° C.-65° C. for 8 hr under vacuum.

Molar Yield: 90%; HPLC Purity >98.0%

Azido impurity: <1 ppm (BDL) obtained from Losartan base having 2100-2500 ppm azido impurity.

### Example-12: Purification of Losartan Free Base

Charged Losartan free base (5.0 g) (having azido impurity at 2100-2500 ppm) in aqueous Potassium hydroxide solution (0.80 g in 25 mL DM water), then sodium borohydride (1.34 g) and TBAB (50 mg) was added to it. Reaction mixture was heated to 1 hr at 80° C.-85° C. The product is isolated after the pH is adjusted to 3.8-4.2 with addition of Aq. HCl in the presence of ethyl acetate. The product is dried initially at 25° C.-30° C. and then at 60° C.-65° C. for 8 hr under vacuum.

Molar Yield: 90%; HPLC Purity >98.0%

Azido impurity: <1 ppm (BDL) obtained from Losartan base having 2100-2500 ppm azido impurity.

The inventors have shown unexpected results in forming pure losartan and losartan potassium that have significantly reduced levels of azido impurities. The inventors have shown that numerous purification methods do not sufficiently reduce levels of the various azido impurities disclosed herein, including by (1) dissolving losartan potassium in water and washing with an organic solvent does not sufficiently reduce the azido impurities; (2) crystallization of losartan potassium from IPA/n-Heptane, IPA or ethyl acetate, methyl isobutyl ketone, ethyl acetate/water/HCl mixture, isopropyl alcohol/water mixture does not sufficiently reduce the azido impurities; and (3) heating of losartan or losartan potassium in aqueous potassium carbonate does not sufficiently reduce the azido impurities. To demonstrate the results of the purification methods according to the invention, the inventors have developed HPLC, LCMS, and LCMS-MS analytical methods specific for detecting and quantifying these impurities. In particular, the inventors have developed and used an HPLC analytical method for determining azido impurities during reaction monitoring and a LCMS or LCMS-MS analytical method for determining azido impurities in the isolated losartan or losartan salt.

The invention also relates to pharmaceutical compositions of losartan or its salts thereof and the process of preparing these compositions that have low levels of azido impurities. The process includes using the losartan or losartan potassium formed as described herein and optionally analyze to determine that the azido content is low and remains in acceptable level. The process includes screening the drug and each excipient for the presence and level of any azido impurities. If the azido impurity level is less than 20 ppm, or preferably less than 10 ppm, or preferably less than 1 ppm, using the drug and excipient in the process of preparation of the pharmaceutical composition. If the azido impurity in the losartan, losartan salt or excipient is too high, e.g., more than 20 ppm, preferably more than 10 ppm, preferably more than 5 ppm, more preferably greater than 1 ppm, not using the losartan, losartan salt or excipient in the pharmaceutical composition. If the azido content is too high in the losartan or losartan salt, the compound can be processed as described herein to lower the level of the azido impurity. In this manner, the resulting pharmaceutical composition can be produced to have a pharmaceutically acceptably low level of any azido impurity. The losartan, losartan salt, excipient and dosage form can be analyzed by LCMS-MS or HPLC, or some combination of both methods, in the process of producing a pharmaceutical composition of losartan or a losartan salt having very low levels of any azido impurity.

Azido Impurities and Impact on Formulation:

The presence of azido impurities adversely affects the safety and shelf life of formulations. The present inventor intensively studied all the critical factors, which can be a cause of azido impurities in the formulation. The present inventor performed the following approaches:
1. In the first method, the present inventors performed experiments and results without applying any control.
2. In the second methodology, the present inventors performed experiments and results by applying all technical efforts to control the content of undesirable genotoxic azido impurities in the formulation.
3. The present inventors performed various testing on each excipient used in the development of formulations.
4. The present inventors also performed various testing on each process step during the manufacturing process to determine the impact of azido impurities. To screen trace levels of impurities in drug/formulation/excipients, a highly sensitive analytical method capable of detecting the impurity at even a PPM level was used, i.e., LCMS or LCMS-MS.
5. The present inventors studied various factors such as: a) screening of all drug sources and excipients being used in the formulation to identify the major contributors for azido impurities; b) identification of alternative sources of the same excipients with negligible levels of azido impurities; c) identification of new excipients.

The following examples relate to pharmaceutical compositions of losartan or a losartan salt and the process of preparing these compositions with the objective of reducing the levels of azido impurities in the compositions.

### Example 13-15

Losartan tablets were prepared by using the quantitative formula as given in Table 7:

TABLE 7
(Quantity/Tablet (% w/w)).

| S. | | | | | |
|---|---|---|---|---|---|
| No. | Ingredient | Function | 13 | 14 | 15 |
| 1 | Losartan or its salts thereof | Active | 0.001-97 | 0.001-50 | 0.001-30 |
| | | Ingredient | | | |
| 2 | Lactose/ | Diluent | 1-95 | 5-95 | 10-95 |
| | Microcrystalline cellulose/ | | | | |
| | Mannitol/Xylitol/Dicalcium | | | | |
| | phosphate/Pre-Gelatinized | | | | |
| | starch/HPC | | | | |
| 3 | Povidone/Hypromellose/ | Binder | 0-30 | 0-20 | 0.1-10 |
| | Starch/Microcrystalline | | | | |
| | cellulose/Pre-Gelatinized | | | | |
| | starch | | | | |
| 4 | Sodium starch glycolate/ | Disintegrant | 0-20 | 0-10 | 0.1-10 |
| | Croscarmellose sodium/ | | | | |
| | Crospovidone/Pre- | | | | |
| | Gelatinized starch/ | | | | |
| | Microcrystalline cellulose/ | | | | |
| | L-HPC | | | | |
| 5 | Colloidal silicon dioxide/ | Glidant | 0.1-5 | 0.1-2 | 0.1-2 |
| | Talc/calcium silicate/ | | | | |
| | Titanium Dioxide | | | | |
| 6 | Magnesium Stearate/Stearic | Lubricant | 0.1-5 | 0.1-3 | 0.1-2 |
| | acid/Sodium stearyl | | | | |
| | fumarate | | | | |

Preferred methods of manufacture: wet granulation, dry granulation, direct compression, and extrusion-spheronization.

### Example 16-17

Losartan tablets were prepared by using the quantitative formula as given in Table 8.

TABLE 8
(Quantity/Tablet (% w/w)).

| S. No. | Ingredient | Function | 16 | 17 |
|---|---|---|---|---|
| 1 | Losartan potassium | Active Ingredient | 33.3 | 32.2 |
| 2 | Lactose | Diluent | 37.0 | 35.7 |
| 3 | Microcrystalline cellulose | Diluent | 16.7 | 16.1 |
| 4 | Pre-Gelatinized starch | Binder/ | 12.0 | 11.6 |
| | | Disintegrant | | |
| 5 | Magnesium Stearate | Lubricant | 1.0 | 1.0 |
| 6 | hypromellose, | Coating Agents | — | 3.4 |
| | hydroxypropyl cellulose, | | | |
| | titanium dioxide, and | | | |
| | solvents | | | |

Preferred methods of manufacture: i) The accurately weighed quantities of active agent and other excipients such as diluent, disintegrant/binder, were sifted through a suitable sieve; ii) The sifted active agent and excipients were mixed using a suitable blender; iii) The sifted blend of step ii) was lubricated, and iv) The lubricated blend was compressed into tablets using suitable punches.

Analytical method: The content of azido impurities in the pharmaceutical composition was analyzed using the LCMS-MS method which comprised the steps of: a) a tablet composition comprising losartan or its salts thereof and at least one or more pharmaceutically acceptable excipients was provided, b) said composition was dissolved in a solvent, c) the amount of formula V in the said sample was analyzed by LCMS-MS method.

### Example 18

Approach 1: Azido Impurities Content in the Formulation without any Control:

The present inventor observed that some marketed tablet samples were found to have higher than permissible levels of azido impurities i.e. more than 100 ppm. As analyzed by the present inventor, some marketed formulations contain a higher amount of undesirable azido impurity ranging from more than 350 ppm to 625 ppm or more. Table 9, below, describes the high presence of azido impurity (formula V) content in formulations (Examples 16-18) when prepared without applying any control in API, composition, excipients, and manufacturing process:

TABLE 9

| S. No. | Formula V Content (ppm) |
|---|---|
| 1 | 393.6 |
| 2 | 419.4 |

Approach 2: Azido Impurities Content in the Formulation while Applying Controls:

Due to the genotoxic side effects associated with azido impurities, the inventor recognized a need to (i) provide stabilized formulations that contain azido impurities within acceptable limits and (ii) exhibit substantially no change in their level of azido impurities upon storage. Various technical studies were carried out by the present inventor to reduce the level of undesirable azido impurities in composition. The API and formulation excipients were tested for azido impurity prior to the formulation process. The basic objective was to use excipients and API free from azido impurities (less than 20 ppm; preferably 0 to 10 PPM), As a result, it has been found that the azido impurities formed during the development of the solid oral composition of losartan can be reduced or substantially completely removed by the selection of suitable API, excipients, their specific amount and ratio, manufacturing process, and process parameters during formulation development.

The azido impurity (formula V) content in the prepared formulation (Examples 16 and 17) was BDL (Below Detection limit) as highlighted in Table 10, following approach 2 while applying controls:

TABLE 10

| S. No. | Example 16 | Example 17 |
|---|---|---|
| 1 | BDL | BDL |

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope, and spirit of this invention.

### Abbreviations Used Herein

THF—tetrahydrofuran
DMF—dimethylformamide
ACN—acetonitrile

### LOD—Limit of Detection

BDL—below detection limit
V—volume
IPA—isopropyl alcohol
DM water—demineralized water
KOH—potassium hydroxide
HCl—hydrochloric acid
NMT—not more than

1. A process for the preparation of Losartan or its pharmaceutically acceptable salts thereof containing less than 10 ppm of any individual azido impurity, wherein the azido impurity is selected from the group consisting of:
5-(4′-(azidomethyl)-[1,1′-biphenyl]-2-yl)-1H-tetrazole,
4′-(azidomethyl)-[1,1′-biphenyl]-2-carbonitrile,
4′-((5-(azidomethyl)-2-butyl-4-chloro-1H-imidazol-1-yl)methyl)-[1,1′-biphenyl]-2-carbonitrile,
5-(4′-((5-(azidomethyl)-2-butyl-4-chloro-1H-imidazol-1-yl)methyl)-[1,1′-biphenyl]-2-yl)-1H-tetrazole,
5-(azidomethyl)-2-butyl-4-chloro-1H-imidazole,
4′-((4-(azidomethyl)-2-butyl-5-chloro-1H-imidazol-1-yl)methyl)-[1,1′-biphenyl]-2-carbonitrile,
5-(4′-((4-(azidomethyl)-2-butyl-5-chloro-1H-imidazol-1-yl)methyl)-[1,1′-biphenyl]-2-yl)-1H-tetrazole, and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole, the process comprising the steps of:
(i) providing a reaction mixture of Losartan or its pharmaceutically acceptable salts thereof in a solvent with an alkali;
(ii) heating the reaction mixture of step (i) in the presence of a reagent, gas or mixture thereof; and
(iii) isolating Losartan or its pharmaceutically acceptable salts thereof,
wherein the reagent is selected from the group consisting of sodium borohydride, sodium borohydride with phase transfer catalyst, lithium borohydride, zinc borohydride, lithium aluminum hydride, glucose, ammonium formate with zinc, ammonium formate with copper, alkyldichloro borane, aryldichloro borane, triethyl borane, and borontrifluoride diethyl etherate with sodium iodide or potassium iodide.

2. The process according to claim 1, wherein the solvent in step (i) is selected from the group consisting of water, an organic solvent and mixtures thereof.

3. The process according to claim 1, wherein the alkali in step (i) is selected from the group consisting of sodium hydroxide, potassium hydroxide, lithium hydroxide, calcium hydroxide and magnesium hydroxide.

4. The process according to claim 1, wherein the alkali in step (i) is sodium hydroxide or potassium hydroxide.

5. The process according to claim 1, wherein the alkali in step (i) is an aqueous alkali.

6. The process according to claim 1, wherein the heating in step (ii) is carried out at temperature from 70° C. to 110° C.

7. The process according to claim 1, wherein the heating in step (ii) is carried out at temperature from 80° C. to 100° C.

8. The process according to claim 1, wherein the heating in step (ii) is carried out for 10 min to 10 hrs.

9. (canceled)

10. The process according to claim 1, wherein the gas in step (ii) is selected from the group comprising of hydrogen gas, nitrogen gas, argon and neon gas.

11. The process according to claim 1, wherein the process comprises the steps of:
(i) providing a reaction mixture of Losartan or its pharmaceutically acceptable salts thereof in a solvent with an alkali;
(ii) heating the reaction mixture of step (i) in the presence of a reagent, gas or mixture thereof;
(iii) adding an organic solvent and adjusting the pH to a pH of 3 to 5 by means of adding an acid to the reaction mixture;
(iv) filtering the reaction mixture of step (iii); and
(v) isolating Losartan or its pharmaceutically acceptable salts thereof,
wherein the reagent is selected from the group consisting of sodium borohydride, sodium borohydride with phase transfer catalyst, lithium borohydride, zinc borohydride, lithium aluminum hydride, glucose, ammonium formate with zinc, ammonium formate with copper, alkyldichloro borane, aryldichloro borane, triethyl borane, and borontrifluoride diethyl etherate with sodium iodide or potassium iodide.

12. The process according to claim 11, wherein the heating in step (ii) is carried out at temperature from 70° C. to 110° C.

13. The process according to claim 11, wherein the heating in step (ii) is carried out at temperature from 80° C. to 100° C.

14. The process according to claim 11, wherein the heating in step (ii) is carried out for 10 min to 10 hrs.

15. (canceled)

16. The process according to claim 11, wherein the gas in step (ii) is selected from the group consisting of hydrogen gas, nitrogen gas, argon and neon gas.

17. A process for the preparation of Losartan or its pharmaceutically acceptable salts thereof containing less than 10 ppm of any individual azido impurity, wherein the azido impurity is selected from the group consisting of:
5-(4′-(azidomethyl)-[1,1′-biphenyl]-2-yl)-1H-tetrazole,
4′-(azidomethyl)-[1,1′-biphenyl]-2-carbonitrile,
4′-((5-(azidomethyl)-2-butyl-4-chloro-1H-imidazol-1-yl)methyl)-[1,1′-biphenyl]-2-carbonitrile,
5-(4′-((5-(azidomethyl)-2-butyl-4-chloro-1H-imidazol-1-yl)methyl)-[1,1′-biphenyl]-2-yl)-1H-tetrazole,
5-(azidomethyl)-2-butyl-4-chloro-1H-imidazole,
4′-((4-(azidomethyl)-2-butyl-5-chloro-1H-imidazol-1-yl)methyl)-[1,1′-biphenyl]-2-carbonitrile,
5-(4′-((4-(azidomethyl)-2-butyl-5-chloro-1H-imidazol-1-yl)methyl)-[1,1′-biphenyl]-2-yl)-1H-tetrazole, and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole, the process comprising the steps of:
(i) providing a reaction mixture of Losartan or its pharmaceutically acceptable salts thereof in a solvent with an alkali;
(ii) heating the reaction mixture in an autoclave; and
(iii) isolating Losartan or its pharmaceutically acceptable salts thereof.

18. The process according to claim 17, wherein the heating in step (ii) is carried out at temperature from 70° C. to 110 C.

19. The process according to claim 17, wherein the heating in step (ii) is carried out for 10 min to 10 hrs.

20. A process for the preparation of Losartan or its pharmaceutically acceptable salts thereof containing less than 10 ppm of any individual azido impurity, wherein the azido impurity is selected from the group consisting of:
5-(4′-(azidomethyl)-[1,1′-biphenyl]-2-yl)-1H-tetrazole,
4′-(azidomethyl)-[1,1′-biphenyl]-2-carbonitrile,
4′-((5-(azidomethyl)-2-butyl-4-chloro-1H-imidazol-1-yl)methyl)-[1,1′-biphenyl]-2-carbonitrile,
5-(4′-((5-(azidomethyl)-2-butyl-4-chloro-1H-imidazol-1-yl)methyl)-[1,1′-biphenyl]-2-yl)-1H-tetrazole,
5-(azidomethyl)-2-butyl-4-chloro-1H-imidazole,
4′-((4-(azidomethyl)-2-butyl-5-chloro-1H-imidazol-1-yl)methyl)-[1,1′-biphenyl]-2-carbonitrile,
5-(4′-((4-(azidomethyl)-2-butyl-5-chloro-1H-imidazol-1-yl)methyl)-[1,1′-biphenyl]-2-yl)-1H-tetrazole, and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole, the process comprising the steps of:
(i) providing a reaction mixture of Losartan or its pharmaceutically acceptable salts thereof in a solvent with an alkali;
(ii) heating the reaction mixture in presence of a reagent, a gas or a mixture thereof; and
(iii) isolating Losartan or its pharmaceutically acceptable salts thereof,
wherein the reagent is selected from the group consisting of glucose and sodium borohydride with tetra butyl ammonium bromide; and
the gas is selected from the group consisting of hydrogen gas, nitrogen gas, argon and neon gas.

21. The process according to claim 1, further comprising:
analyzing at least one pharmaceutically acceptable excipient for a level of any one of the azido impurities; and
preparing a pharmaceutical composition of the losartan or losartan salt with the at least one pharmaceutically acceptable excipient if the level of any individual azido impurity in the excipient is less than 10 ppm.

22. The process according to claim 17, further comprising:
analyzing at least one pharmaceutically acceptable excipient for a level of any one of the azido impurities; and
preparing a pharmaceutical composition of the losartan or losartan salt with the at least one pharmaceutically acceptable excipient if the level of any individual azido impurity in the excipient is less than 10 ppm.

23. The process according to claim 20, further comprising:
analyzing at least one pharmaceutically acceptable excipient for a level of any one of the azido impurities; and
preparing a pharmaceutical composition of the losartan or losartan salt with the at least one pharmaceutically acceptable excipient if the level of any individual azido impurity in the excipient is less than 10 ppm.

24. A process for the preparation of Losartan or its pharmaceutically acceptable salts thereof containing less than 10 ppm of any individual azido impurity, wherein the azido impurity is selected from the group consisting of:
5-(4′-(azidomethyl)-[1,1′-biphenyl]-2-yl)-1H-tetrazole,
4′-(azidomethyl)-[1,1′-biphenyl]-2-carbonitrile,
4′-((5-(azidomethyl)-2-butyl-4-chloro-1H-imidazol-1-yl)methyl)-[1,1′-biphenyl]-2-carbonitrile,
5-(4′-((5-(azidomethyl)-2-butyl-4-chloro-1H-imidazol-1-yl)methyl)-[1,1′-biphenyl]-2-yl)-1H-tetrazole,
5-(azidomethyl)-2-butyl-4-chloro-1H-imidazole,
4′-((4-(azidomethyl)-2-butyl-5-chloro-1H-imidazol-1-yl)methyl)-[1,1′-biphenyl]-2-carbonitrile,
5-(4′-((4-(azidomethyl)-2-butyl-5-chloro-1H-imidazol-1-yl)methyl)-[1,1′-biphenyl]-2-yl)-1H-tetrazole, and
1-((1-((2′-(1H-tetrazol-5-yl)-[1,1′-biphenyl]-4-yl)methyl)-2-butyl-4-chloro-1H-imidazol-5-yl)methyl)-5-(4′-((5-(azidomethyl)-2-butyl-4-chloro-1H-imidazol-1-yl)methyl)-[1,1′-biphenyl]-2-yl)-1H-tetrazole, the process comprising the steps of:
(i) providing a reaction mixture of Losartan or its pharmaceutically acceptable salts thereof in a solvent with an alkali;
(ii) heating the reaction mixture of step (i) in the presence of a reagent, gas or mixture thereof; and
(iii) isolating Losartan or its pharmaceutically acceptable salts thereof,
wherein the alkali in step (i) is selected from the group consisting of sodium hydroxide, potassium hydroxide, lithium hydroxide, calcium hydroxide and magnesium hydroxide.

25. The process according to claim 24, wherein the reagent is selected from the group consisting of sodium borohydride, sodium borohydride with phase transfer catalyst, lithium borohydride, zinc borohydride, lithium aluminum hydride, glucose, ammonium formate with zinc, ammonium formate with copper, alkyldichloro borane, aryldichloro borane, triethyl borane, and borontrifluoride diethyl etherate with sodium iodide or potassium iodide.
